(19) United States
(12) Patent Application Publication
(10) Pub. No.: US 2006/0111778 A1
(43) Pub. Date: May 25, 2006

(54) Methods of promoting healing of cartilage defects and method of causing stem cells to differentiate by the articular chondrocyte pathway

(76) Inventor: Alexander E. Michalow, Bourbonnais, IL (US)

Correspondence Address: GARDNER CARTON & DOUGLAS LLP;ATTN: PATENT DOCKET DEPT., 191 N. WACKER DRIVE, SUITE 3700, CHICAGO, IL, 60606, US

(21) Appl. No.: 11/262,080

(22) Filed: Oct. 27, 2005

Related U.S. Application Data
(60) Provisional application No. 60/623,158, filed on Oct. 29, 2004.
(60) Provisional application No. 60/720,304, filed on Sep. 23, 2005.

(51) Int. Cl. A61F 2/28; C12N 5/08

(52) U.S. Cl. 623/14.12; 623/23.63; 435/395

(57) ABSTRACT

Methods of promoting healing of a cartilage defect in a region of cartilage, which comprises the defect and which may further comprise stem cells, and methods of promoting healing of a cartilage defect in a region of cartilage, which comprises the defect and an implant comprising cartilage scaffold or a cartilage graft, which methods comprise contacting the region with various combinations of cartilage fragments, a growth factor, a partially synthesized extracellular matrix, a scaffold, an implant comprising cartilage scaffold, an implant comprising a cartilage graft, stem cells, chondrocytes, a proteoglycan, an anti-oxidant, a collagen precursor, a vitamin, a mineral, and/or a cartilage-degrading enzyme; and a method of causing stem cells to differentiate by the articular chondrocyte pathway comprising contacting the stem cells with a compound comprising an active alcohol moiety.

### CROSS-REFERENCE TO RELATED PATENT APPLICATIONS

This patent application claims the benefit of U.S. Provisional Patent Application No. 60/623,158, filed Oct. 29, 2004, and U.S. Provisional Patent Application No. 60/720,304, filed Sep. 23, 2005, the entire contents of which are herein incorporated by reference.

### TECHNICAL FIELD OF THE INVENTION

The present invention relates to methods of using cartilage fragments, alone or in combination with other agents, to promote healing of cartilage defects, and to a method of using alcohol to cause stem cells to differentiate by the articular chondrocyte pathway.

### BACKGROUND OF THE INVENTION

Hyaline cartilage (referred to herein as ‘cartilage’) is that cartilage which is present in all joints that articulate against each other. It serves two main functions. It acts to absorb and/or dissipate forces across the joint, and it is responsible for the low friction that is present in all articulating joints.

Cartilage is made up of cells called chondrocytes and an extracellular matrix (ECM). The ECM consists of proteoglycans (PGs) and collagen, along with numerous other proteins that all serve certain functions, and an abundance of water. The PGs are organized into large molecules called aggrecan. Aggrecan consists of a backbone of a long-chain hyaluronic acid polymer, which has multiple protein cores attached to it. Each protein core has numerous PG chains, which are attached to it and which lie adjacent to each other. The PG chains have an overall negative charge and attract water. The PGs are generally responsible for cushioning compressive forces that are put onto a joint.

The primary collagen in cartilage is type II collagen, which makes up 90% of all of the collagen (in an adult). Cartilage types VI, IX and XI make up the majority of the other collagens. Collagen acts to absorb tension and shear forces that act upon a joint. The collagen acts in concert with the PGs to dissipate compression, tension and shear.

The structure of cartilage is non-homogeneous. There are several layers. The outer layer is the tangential (or superficial) zone, followed by the transitional zone, the radial (or deep) zone, the tidemark (signifies the transition between non-mineralized and mineralized zones), and the calcified cartilage zone. The collagen structure and PGs differ in their alignment and concentration through the different zones. The radial and tangential zones are connected by the collagen network. The collagen fibers form an arcade with the base at the calcified cartilage zone and the top of the arcade at or near the tangential zone. The calcified cartilage zone connects to the underlying bone by interdigitating spicules of bone. The gross structure of cartilage PGs and collagen is important in enabling it to dissipate forces and, at the same time, be responsible for low-friction joint motion.

Chondrocytes are spread throughout the cartilage zones, although in varying concentrations and in varying alignment for each of the zones. In the tangential zones the chondrocytes are more tightly packed and are arranged rather spuriously. In the radial zones they assume a columnar pattern.

The structure of the ECM is further divided with respect to the chondrocytes. There are three zones around the chondrocytes called the pericellular matrix, the territorial matrix, and the inter-territorial matrix. Chondrocytes maintain these extra-cellular matrices.

Just as for other tissues in the body there is a continuous breakdown and buildup of cartilage tissue. This metabolism is balanced in the normal joint. However, given that the half-life of collagen II is 100 years and the half-life of aggrecan is 1-2 years, it is common for it to take 6-18 months of increased turnover for healing to occur in the ECM after an injury occurs.

Any disruption causes first an increase in the breakdown, then a buildup of the disrupted cartilage. When the disruption is not extensive, the cartilage can remodel itself back to normal. Any loss, significant disruption, and/or inability to restore this architecture results in poor mechanical properties. Over time these poor mechanical properties of fibrous cartilage result in its gradual breakdown, which leads to osteoarthritis.

When hyaline cartilage is disrupted more extensively, such as when defects develop from trauma or other causes, it is sometimes not possible for complete healing to occur. This is at least partially due to the low metabolic rate of the chondrocytes, which are anaerobic cells. Furthermore, the healing response in humans, in general, is to form scar tissue. Scar tissue has an abundance of type III collagen. Type III collagen has a rather uncoordinated structure as compared to type II collagen in cartilage, or type I cartilage in other tissues, such as skin, bone, ligament, or tendon. Due to the poor organization of type III collagen, it is associated with poor mechanical properties.

Because large defects are unable to repair themselves with a normal hyaline cartilage ECM structure it is generally recommended that cartilage defects are repaired. To date, however, there has not been developed an optimal manner by which to repair cartilage defects.

The repair of cartilage defects includes numerous different techniques. More traditional methods include arthroscopic abrasion arthroplasty and microfracture. Abrasion arthroplasty and the microfracture technique are advantageous in that the entire procedure can be done at one arthroscopic setting with relatively little damage to surrounding normal cartilage tissue. The disadvantage of such methods is that only fibrous cartilage is formed. In addition, these techniques generally are effective and reasonably successful only for small defects, i.e., less than 1 cm², and in the younger patient.

For larger defects, and especially those that involve the underlying subchondral bone, the use of osteochondral grafts is advocated. This includes the use of autologous grafts, called the OATS (osteochondral autograft transfer system) procedure. This generally involves the transfer of bone and cartilage from an area of uninvolved cartilage to the damaged area. It can include the use of a single large piece of bone and cartilage. It can also involve the use of several smaller autologous grafts in a procedure called the mosaicplasty or the use of bone and cartilage paste that is manually crushed at the time of surgery (U.S. Pat. No. 6,110,209). Mosaicplasty and the OATS procedure are advantageous in that at least some normal hyaline cartilage is present in the defect. Furthermore, the chondrocytes remain viable, and they are the patient's own cells. Thus, there is no problem with graft rejection or the need to supply cells into the graft. However, fibrous cartilage tends to form at the borders. Also, while long-term results at 5 years are favorable, there is still the potential for the development of osteoarthritis. Furthermore, these procedures are technically difficult when one attempts to obtain a smooth cartilage border, and any graft irregularity leads to failure. Other potential problems include graft subsidence, harvest site degeneration; etc. Furthermore, although these methods can be done arthroscopically, many times an arthrotomy is needed.

Another option is the use of an allograft osteochondral graft from a cadaver. Although these have reasonably good results in the long term, they are problematic in that they require that one have a tissue bank and the ready availability of fresh allogeneic tissue, which is available in only very few centers. In addition, even though there is no cell-mediated immune response, the body does launch a humoral immune response, thereby rendering future blood transfusions or other transplants problematic.

Whenever one is concerned with tissue healing, there is the need to consider what cell type will be responsible for the healing process. For cartilage healing one can rely on either chondrocytes or stem cells. When chondrocytes are used, they are generally in vitro culture-expanded first, prior to reimplantation into a defect, in order to obtain large numbers of these cells. U.S. Pat. No. 6,200,606 describes a manner by which to isolate chondrocyte precursor cells, which then can be used for cartilage repair, with or without a carrier material and without the need for in vitro culturing. Stem cells may either come from the underlying bone marrow, as occurs with the micro-fracture technique, or they can be harvested from a patient's bone marrow at the iliac crest and subsequently inserted into a cartilage defect, with or without in vitro cell expansion.

When culture-expanded chondrocytes are reimplanted into a cartilage defect, such a procedure is called autologous chondrocyte implantation or ACI (Vacanti et al., Int'l Pat. App. Pub. No. WO 90/12603; and Brittberg et al., Treatment of Deep Cartilage Defects in the Knee with Autologous Chondrocyte Transplantaton, New Engl. J. Med. 331: 889-895 (1994)). In this procedure knee arthroscopy is performed to identify and biopsy healthy cartilage tissue. Chondrocytes are separated from the biopsied tissue and cultivated in culture media for 14-21 days. An arthrotomy is subsequently performed, and the cartilage lesion is excised up to the normal surrounding tissue. The cultured chondrocytes are then injected under a periosteal flap, which is sutured around the borders of the defect.

Numerous scaffolds have been developed for insertion into cartilage defects. See the review article in Biomaterials 21 (2000).

Some scaffolds are acellular and depend on the in-migration of surrounding cells to vitalize the implant. Acellular scaffolds that can be inserted into a defect are described in U.S. Pat. Nos. 5,368,858; 5,624,463; 5,866,165; 5,876,444; and 5,972,385.

Other scaffolds are mixed with chondrogenic cells (chondrocytes or stem cells) and inserted into a defect. Scaffolds that are mixed with cells and then inserted into a cartilage defect are described in U.S. Pat. Nos. 4,642,120; 4,904,259; and 6,623,963.

Other scaffolds are cultured in vitro to form a partial cartilage ECM for implantation into defects where they act as three-dimensional attachment sites for cells. The in vitro culturing of chondrogenic cells within a matrix to generate a partially synthesized cartilage graft for insertion into a defect is described in U.S. Pat. Nos. 5,736,372; 5,866,415; 5,902,741; 6,171,610; 6,183,737; 6,197,061; 6,235,316; 6,264,701; 6,294,202; 6,387,693; 6,451,060; 6,623,963; 6,645,764; 6,703,041; and 6,852,331.

The use of collagen and/or any other material as a scaffold is described in U.S. Pat. Nos. 4,846,835; 5,842,477; 5,876,444; 5,902,741; 5,922,028; 6,176,880 (intestinal submucosa); 5,904,717; 5,939,323 (hyaluronan); 6,080,194; 6,326,029; 6,378,527 (chitosan); 6,444,222; and 6,676,969.

The use of matrices or scaffolds that are either acellular or have had cells added to them is problematic in that they generally require many months to be degraded, while at the same time being replaced by normal cartilage ECM. If one could accelerate the degradation of added scaffold and, at the same time, accelerate the synthesis of cartilage ECM, the time to graft maturation would be shortened.

U.S. Pat. No. 6,677,306 describes the use of amelogenin peptides for inducing chondrogenesis, but no specific matrix is described. U.S. Pat. No. 6,251,143 describes a cartilage repair unit of bio-absorbable material. The use of hyaluronan is described in Int'l Pat. App. Pub. Nos. WO 99/61080, WO 99/65534, and WO 02/053201, whereas the use of type I collagen is described in WO 03/080141, and the use of type II collagen is described in WO 02/089866. The in vitro production of cartilage tissue is described in WO 2004/104188, whereas the regeneration of connective tissue is described in WO 2005/042048.

There are several ongoing clinical trials in which in vitro partially synthesized cartilage grafts are being tested for the repair of cartilage defects. One such system is termed CaReS (cartilage repair system) by ARS Arthro AG (Germany). In their proprietary technique chondrocytes are cultured in a three-dimentional (3-D) scaffold made out of type I collagen hydrogel, which is obtained from rat tail tendon. The culturing technique results in a partially synthesized cartilage ECM graft, which is implanted into a cartilage defect.

Another such system is called Carticel II®. In this technique chondrocytes are cultured in a collagen II matrix until a partially synthesized cartilage ECM is produced.

Yet another such system has been developed by Fidia (Italy). In this technique chondrocytes are cultured in a hyaluronic acid polymeric matrix until partially synthesized cartilage ECM is produced.

The above prior art utilizes chondrocytes as the primary cell, although several of the above patents also advocate the use of stem cells with certain scaffold materials. The use of stem cells, most commonly mesenchymal stem cells (MSCs), is also advocated for the repair of cartilage defects. U.S. Pat. No. 6,174,333 describes the use of a collagen gel matrix with MSCs in order to regenerate cartilage. U.S. Pat. No. 6,214,369 describes a method involving the implantation of cartilaginous matrix, which is produced by MSCs embedded in a biodegradable polymeric matrix of natural or synthetic polymers. Either the polymeric matrix is (i) seeded with MSCs, cultured in vitro and then implanted, (ii) seeded with MSCs and immediately implanted, or (iii) implanted and then seeded with MSCs. U.S. Pat. No. 6,355,239 discloses the use of a therapeutically effective amount of MSCs for the treatment of a cartilaginous defect. The problem with merely administering MSCs into a joint is that fibrous cartilage, rather than hyaline cartilage, is formed (communication of Frank Barry, Director, Arthritis Research, Osiris Corp., Baltimore, Md., at “Joint Preservation—Treatment of the Knee” conference, Williamsburg, Va. (2002); see, also, Wakitani et al., JBJS 76-A(4): 570-592 (1994)).

The advantage of using chondrocytes is that they are programmed to synthesize ECM components. There is no need to induce these cells to become chondrocytes. Stem cells, on the contrary, need to be induced to differentiate into chondrocytes before they will synthesize cartilage ECM.

The disadvantage of using chondrocytes is that they need to be culture-expanded for most applications in order to obtain an adequate number of these cells to heal a cartilage defect. In addition, in order to culture-expand chondrocytes, a surgical procedure is performed where cartilage fragments are biopsied. The fragments are then enzymatically separated from their surrounding matrix and subsequently the cellular proliferation and expansion process takes place. The disadvantage of this procedure is that two intra-articular surgical procedures are needed—one to obtain the cells and another to re-insert the cells, either alone or within a partially synthesized cartilage ECM, into the cartilage defect.

The advantage of using stem cells is that one can harvest the cells from a patient, i.e., pelvic iliac crest, which can be done under local anesthesia and obviates the need for performing an intra-articular biopsy. Also, there is the potential for the use of allogeneic stem cells, e.g., mesenchymal or other stem cells. Such cells do not incite an immune response when inserted into a non-HLA-matched recipient. The use of allogeneic cells obviates the need for any prior harvesting procedure for cartilage repair.

Growth factors have a significant stimulatory and/or chondrocyte induction effect. Such growth factors include insulin-like growth factor (IGF-1), fibroblast growth factor (FGF), transforming growth factor β (TGF-β), including types 1, 2, and 3, hepatocyte growth factor (HGF), platelet-derived growth factor (PDGF), Indian hedgehog (Ihh), bone morphogenic protein (BMP), interleukin-1 receptor antagonist (IL-1ra) (Hickey, Am. J. Ortho. February 2003: 70-76), and growth hormone (GH). The use of TGF-β1 and/or TGF-β2 as growth stimulants for chondrocytes in a cell expansion process is described in U.S. Pat. No. 6,150,163. The use of TGF for three-dimensional cultures of cartilage in vitro is described in U.S. Pat. No. 5,902,741.

The methods for repairing or replacing cartilage described in the aforementioned U.S. patents suffer from various disadvantages and limitations. For example, synthetic scaffolds are prone to fibrous cartilage formation. Furthermore, many scaffolds are too weak to withstand the mechanical stresses to which they are subjected in the intra-articular environment. Collagen grafts, while commonly used, also are prone to fibrous cartilage formation. Many methods require the suturing of a patch over the implant site, and the suturing breaks down local normal cartilage. Furthermore, it has been shown in a goat model that delamination of the patch can approach 100% of the patches when unrestricted joint motion is allowed. Even with immobilization, the rate of flap survival is only 67% and 95% for periosteal and fascial flaps, respectively. Furthermore, when cells are injected underneath a patch, only a small percent actually survive, i.e., 8%. Indeed, the biggest hurdle in hyaline cartilage repair is developing a manner/method by which to induce cells at the site of a cartilaginous defect to synthesize normal hyaline ECM, while at the same time inhibiting the formation of fibrous cartilage.

An optimal cartilage repair method would include a single surgical procedure, result in the formation of normal hyaline cartilage, and incorporate the cartilage graft with surrounding cartilage tissue rapidly and seamlessly. In addition, the formation of normal cartilage ECM would inhibit/minimize fibrous cartilage formation.

The use of stem cells can more readily meet these requirements than does the use of autologous chondrocytes. Stem cells, however, need to differentiate into chondrocytes before they can heal a cartilage defect. To date an optimal manner by which to induce stem cells to differentiate into chondrocytes has not been developed.

In view of the foregoing, it is an object of the present invention to provide methods of repairing hyaline cartilage defects that overcome some of the disadvantages and limitations of currently available repair methods. It is another object of the present invention to provide a method of causing stem cells to differentiate by the articular chondrocyte pathway so that they can heal a cartilage defect. These and other objects and advantages, as well as additional inventive features, will become apparent from the detailed description provided herein.

### BRIEF SUMMARY OF THE INVENTION

The present invention provides a method of promoting healing of a cartilage defect in a region of cartilage, which comprises the defect and stem cells. The method comprises contacting the region with (i) cartilage fragments, (ii) cartilage fragments and a partially synthesized ECM, or (iii) cartilage fragments, a partially synthesized ECM, and a scaffold. The method preferably further comprises contacting the region with at least one growth factor. The cartilage fragments and the at least one growth factor induce the stem cells to differentiate into chondrocytes, thereby promoting healing of the cartilage defect. The method can further comprise simultaneously or sequentially, in either order, contacting the region with stem cells, chondrocytes, a proteoglycan, an anti-oxidant, a collagen precursor, a vitamin, a mineral, and/or a cartilage-degrading enzyme.

The present invention further provides a method of promoting healing of a cartilage defect in a region of cartilage, which comprises the defect and an implant comprising cartilage scaffold. The method comprises contacting the region with (i) cartilage fragments, (ii) cartilage fragments and a partially synthesized ECM, or (iii) cartilage fragments, a partially synthesized ECM, and a scaffold. The method preferably further comprises contacting the region with at least one growth factor. The cartilage fragments promote degradation of the cartilage scaffold in the implant, thereby promoting healing of the cartilage defect. The method can further comprise simultaneously or sequentially, in either order, contacting the region with stem cells, chondrocytes, a proteoglycan, an anti-oxidant, a collagen precursor, a vitamin, a mineral, and/or a cartilage-degrading enzyme.

Still further provided is a method of promoting healing of a cartilage defect in a region of cartilage, which comprises the defect. The method comprises (i) contacting the region with an implant comprising cartilage scaffold, cartilage fragments, and, optionally, a collagen precursor, or (ii) simultaneously or sequentially, in either order, contacting the region with (a) (i′) cartilage fragments, (ii′) cartilage fragments and a partially synthesized ECM, or (iii′) cartilage fragments, a partially synthesized ECM, and a scaffold, and (b) an implant comprising cartilage scaffold. The method preferably further comprises contacting the region with at least one growth factor in (ii). The cartilage fragments promote degradation of the cartilage scaffold in the implant, thereby promoting healing of the cartilage defect. The method can further comprise simultaneously or sequentially, in either order, contacting the region with stem cells, chondrocytes, a proteoglycan, an anti-oxidant, a collagen precursor, a vitamin, a mineral, and/or a cartilage-degrading enzyme.

Even still further provided is a method of promoting healing of a cartilage defect in a region of cartilage, which comprises the defect and an implant comprising a cartilage graft. The method comprises contacting the region with (i) cartilage fragments, (ii) cartilage fragments and a partially synthesized ECM, or (iii) cartilage fragments, a partially synthesized ECM, and a scaffold. The method preferably further comprises contacting the region with at least one growth factor. The cartilage fragments promote incorporation of the cartilage graft into adjacent cartilage in the region, thereby promoting healing of the cartilage defect. The method can further comprise simultaneously or sequentially, in either order, contacting the region with stem cells, chondrocytes, a proteoglycan, an anti-oxidant, a collagen precursor, a vitamin, a mineral, and/or a cartilage-degrading enzyme.

Yet even still further provided is a method of promoting healing of a cartilage defect in a region of cartilage, which comprises the defect. The method comprises (i) contacting the region with an implant comprising a cartilage graft and cartilage fragments or (ii) simultaneously or sequentially, in either order, contacting the region with (a) (i′) cartilage fragments, (ii′) cartilage fragments and a partially synthesized ECM, or (iii′) cartilage fragments, a partially synthesized ECM, and a scaffold, and (b) an implant comprising cartilage scaffold. The method preferably further comprises contacting the region with at least one growth factor in (ii). The cartilage fragments promote incorporation of the cartilage graft into adjacent cartilage in the region, thereby promoting healing of the cartilage defect. The method can further comprise simultaneously or sequentially, in either order, contacting the region with stem cells, chondrocytes, a proteoglycan, an anti-oxidant, a collagen precursor, a vitamin, a mineral, and/or a cartilage-degrading enzyme.

A method of causing stem cells to differentiate by the articular chondrocyte pathway is also provided. The method comprises contacting the stem cells with alcohol, whereupon the stem cells differentiate by the articular chondrocyte pathway.

### DETAILED DESCRIPTION OF THE INVENTION

The present invention provides a method of promoting healing of a cartilage defect in a region of cartilage, which comprises the defect and stem cells. The method comprises contacting the region with (i) cartilage fragments, (ii) cartilage fragments and a partially synthesized ECM, or (iii) cartilage fragments, a partially synthesized ECM, and a scaffold. The method preferably further comprises contacting the region with at least one growth factor. The cartilage fragments induce the stem cells to differentiate into chondrocytes, thereby promoting healing of the cartilage defect by the synthesis of cartilage. The chondrocytes begin to synthesize normal hyaline cartilage ECM.

The present invention further provides a method of promoting healing of a cartilage defect in a region of cartilage, which comprises the defect and an implant comprising cartilage scaffold. The method comprises contacting the region with (i) cartilage fragments, (ii) cartilage fragments and a partially synthesized ECM, or (iii) cartilage fragments, a partially synthesized ECM, and a scaffold. The method preferably further comprises contacting the region with at least one growth factor. The cartilage fragments promote degradation of the cartilage scaffold in the implant, thereby promoting healing of the cartilage defect.

Still further provided is a method of promoting healing of a cartilage defect in a region of cartilage, which comprises the defect. The method comprises (i) contacting the region with an implant comprising cartilage scaffold and cartilage fragments, or (ii) simultaneously or sequentially, in either order, contacting the region with (a) (i′) cartilage fragments, (ii′) cartilage fragments and a partially synthesized ECM, or (iii′) cartilage fragments, a partially synthesized ECM, and a scaffold, and (b) an implant comprising cartilage scaffold. The method preferably further comprises contacting the region with at least one growth factor in (ii). The cartilage fragments promote degradation of the cartilage scaffold in the implant, thereby promoting healing of the cartilage defect.

Even still further provided is a method of promoting healing of a cartilage defect in a region of cartilage, which comprises the defect and an implant comprising a cartilage graft. The method comprises contacting the region with (i) cartilage fragments, (ii) cartilage fragments and a partially synthesized ECM, or (iii) cartilage fragments, a partially synthesized ECM, and a scaffold. The method preferably further comprises contacting the region with at least one growth factor. The cartilage fragments promote incorporation of the cartilage graft into adjacent cartilage in the region, thereby promoting healing of the cartilage defect.

Yet even still further provided is a method of promoting healing of a cartilage defect in a region of cartilage, which comprises the defect. The method comprises (i) contacting the region with an implant comprising a cartilage graft and cartilage fragments or (ii) simultaneously or sequentially, in either order, contacting the region with (a) (i′) cartilage fragments, (ii′) cartilage fragments and a partially synthesized ECM, or (iii′) cartilage fragments, a partially synthesized ECM, and a scaffold, and (b) an implant comprising cartilage scaffold. The method preferably further comprises contacting the region with at least one growth factor in (ii). The cartilage fragments promote incorporation of the cartilage graft into adjacent cartilage in the region, thereby promoting healing of the cartilage defect.

With respect to the above methods, the defect can be full-thickness or partial-thickness, e.g., a crack, a crevice, a mild fibrillation, a flap tear, or an excavated defect. The cartilage can be autologous, allogeneic, or xenogeneic (referred to collectively herein as “cartilage” or “cartilaginous”). Xenogeneic cartilage must be rendered non-immunogenic prior to use in accordance with methods known in the art (see, e.g., U.S. Pat. No. 6,049,025).

The cartilage fragments are prepared by mechanical disruption of pieces of cartilage, such as harvested pieces of cartilage, into smaller fragments. For example, large harvested pieces of cartilage can be mechanically disrupted by cutting, morselizing, grating, grinding, homogenizing, or pulverizing. Preferably, the pieces are rendered less pliable, i.e., more brittle, such as by freezing, e.g., at −30 to −70° C., prior to mechanical disruption. Freezing also devitalizes the cartilage, i.e., kills the cells contained within the cartilage by cellular lysis resulting from freezing at low temperatures and subsequently thawing. Multiple freeze-thaw cycles can be used to ensure more complete cellular lysis. This is especially important for use of allogeneic or xenogeneic cartilage so that the cartilage loses its cellular immunogenic properties. Alternatively, allogeneic or xenogeneic cartilage can be contacted with an apoptotic agent, which, subsequently, must be removed from the fragments. Cell lysis is not important when one uses autologous cartilage.

The cartilage fragments are preferably about 10μ-3 mm in size, more preferably, about 50μ-1 mm in size, and most preferably, about 50-250μ in size. Preferably, the fragments are suspended in medium in weight/volume of about 1-50%, more preferably about 2-25%, and most preferably about 2.5-10%.

The defect can be contacted with the cartilage fragments; etc. using any suitable technique or combination of techniques as is known in the art. See, for example, the Examples set forth herein.

The at least one growth factor can be any suitable growth factor, e.g., a growth factor important for articular cartilage repair (see, e.g., Hickey et al., Am. J. Ortho. February 2003: 70-76). Examples of suitable growth factors include, but are not limited to, transforming growth factor (TGF)-β, such as TGF-β₁, TGF-β₂, or TGF-β₃, insulin-like growth factor (IGF-1), fibroblast growth factor (FGF), hepatocyte growth factor (HGF), platelet-derived growth factor (PDGF), Indian hedgehog (Ihh), bone morphogenic protein (BMP), interleukin-1 receptor antagonist (IL-1ra), and growth hormone (GH).

The cartilage fragments can be optionally mixed with proteoglycans. Examples of proteoglycans include, but are not limited to, hyaluronic acid, chondroitin sulfate, glucosamine sulfate, keratin sulfate, dermatan sulfate, and galactosamine. Synthetic alternatives also can be used. Proteoglycans can be added at a concentration of about 1-50%, preferably about 50-10%. Such factors have stimulating or protecting effects on chondrocytes.

The cartilage fragments also can be contacted with vitamins and/or minerals. Vitamins and minerals are known in the art.

The cartilage fragments also can be contacted with any suitable collagen precursor. Examples include, but are not limited to, amino acids (e.g., proline, hydroxyl-proline, or glycine), gelfoam, and gelfoam powder.

A partially synthesized ECM can be generated by any suitable method. For example, chondrocytes can be cultured for several days up to 1-2 weeks (see, e.g., Pollack, 1975, in “Readings in Mammalian Cell Culture,” Cold Spring Harbor Laboratory Press, Cold Spring Harbor), after which an early ECM is produced. After this early expansion of cells, the mixture of cartilage fragments and chondrocytes is added to another container. Enough of the mixture is added so as to make a 2-mm thick graft. The cell-cartilage mixture is then intermittently irrigated with nutrients; etc. After several weeks up to 4-6 weeks of culture, a partially synthesized cartilage ECM is formed. The texture of this material is softer and more gel-like than that of mature cartilage. Thus, the structure is rather pliable. It can be removed from its culture container by a non-penetrating instrument. The graft then can be implanted into a hyaline cartilage defect or temporarily frozen for future use.

The ECM can be mechanically compressed. Such compression significantly affects the metabolic activity of chondrocytes (Guilak et al., J. Biomech. 33: 1663-1673 (2000)). Intermittent hydrostatic pressure or fluid flow up-regulates the sox9 pathway. The use of mechanical stimuli and/or fluid flow in chondrocyte cultures is described in U.S. Pat. Nos. 5,928,945; 6,037,141; and 6,060,306.

The scaffold can comprise collagen I, collagen II, hyaluronan, or any other natural or synthetic polymer that can support a three-dimensional dispersion of stem cells and chondrocytes. The scaffold can be cellular or acellular. Examples of such additives include, but are not limited to, polyglycolic acid, polylactic acid, alginate, polydioxane, polyester, protein hydrogels, fibrin clot, and various combinations of the foregoing.

The above methods also can further comprise simultaneously or sequentially, in either order, contacting the region with chondrocytes or stem cells, such as dedifferentiated chondrocytes, embryonic stem cells, placental stem cells, mesenchymal stem cells, multi-potent adult progenitor cells, undifferentiated adipose stem cells, undifferentiated fibrocytes, and any undifferentiated cell with the potential to differentiate into a chondrocyte. Such cells can be autologous, allogeneic or xenogeneic and can be culture-expanded in accordance with methods known in the art (see, e.g., Pollack, supra). Preferably, the stem cells have been contacted with alcohol as described herein.

The cartilage fragments, alone or in further combination with chondrocytes or stem cells, can be stabilized by a biological glue. An example of a suitable glue is fibrin. If desired, the glue can be added to the region after the cartilage fragments or the cartilage fragments in combination with the chondrocytes/stem cells and before or after an implant. Other stabilization methods, such as the use of staples or sutures, either one of which can be combined with a covering patch, also can be employed.

The above methods can further comprise contacting the region with an anti-oxidant. Examples of suitable anti-oxidants include, but are not limited to, superoxide dismutase (SOD; preferably in combination with manganese), glycyl-1-histidyl-1-lysine:copper(II) (GHL-Cu), tocopherol, selenium, and ascorbate (preferably in combination with manganese and magnesium). The anti-oxidant helps reduce the presence of oxygen, which, in turn, promotes chondrocyte differentiation and inhibits fibrous tissue formation. Ascorbate is also a co-factor for collagen synthesis.

The above methods can further comprise contacting the region with a cartilage-degrading enzyme, such as collagenase, hyaluronidase, or chondroitinase, in order to partially degrade the edges of the cartilage defect and thereby accelerate the incorporation of an implant. Alternatively, the added chondrocytes or stem cells can be induced to synthesize such enzymes.

The implant can be a cartilage graft, such as a partially synthesized cartilage ECM graft, a scaffold, a mosaicplasty graft, an autologous/allogeneic osteochondral graft, and the like. Alternatively, the implant can be synthetic.

Any suitable method of “contacting” the above components to the region can be used. Such methods are known in the art.

The present invention further provides a method of causing stem cells to differentiate by the articular chondrocyte pathway. The method comprises contacting the stem cells with a compound comprising an active alcohol moiety, whereupon the stem cells differentiate by the articular chondrocyte pathway. Examples of suitable compounds include, but are not limited to, methanol, ethanol, propanol, tert-butanol, or a pharmacologically active salt or analogue thereof, alone or in combination with a carrier therefor. The alcohol is in the form of a solution comprising about 0.5-3% alcohol, such as a solution comprising about 1-2.5% alcohol. This method can be combined with any of the above methods.

For example, stem cells can be cultured in vitro, contacted with a dilute alcohol solution, and then contacted with cartilage fragments. The stem cells can be harvested at the time of surgery for immediate insertion into a cartilage defect, preferably as part of a scaffold. A growth factor is preferably added. An anti-oxidant is optionally added. The induced chondrocytes can be cultured in the presence of a scaffold until a partially synthesized ECM is formed. Scaffolds are as described above. Intermittent hydrostatic pressurization or shear fluid flow can be applied during culture.

Stem cells can be harvested in accordance with methods known in the art. See, e.g., U.S. Pat. No. 6,200,606. The stem cells can be fresh or culture-expanded in vitro. Standard culture expansion techniques for chondrocytes are known in the art. See, e.g., Pollack, “Readings in Mammalian Cell Culture,” Cold Spring Harbor Laboratory Press, Cold Spring Harbor, 1975. After several days up to 1-2 weeks of initial culture expansion, the expanded, de-differentiated chondrocytes are added to a solution of cartilage fragments. After cell-fragment binding takes place, excess fluid is drained. A scaffold, which preferably contains growth factors, anti-oxidants, vitamins, minerals, and other nutrients, is added. The thickness of the mixture can vary from about 2-10 mm. Grafts meant for the femoral condyles are generally about 2-6 mm thick. Grafts for the femoral trochlea are generally about 3-8 mm thick, whereas grafts for the patella are generally about 4-10 mm thick. After about 1-6 weeks of culture, a partially synthesized cartilage ECM is formed. The texture of this material is softer and more gel-like than that of mature cartilage. It is rather pliable and can be removed from its culture contained by a non-penetrating instrument. The graft then can be used for implantation into a hyaline cartilage defect and preferably adhered to the defect with glue, such as fibrin, and/or a patch. Alternatively, the graft can be temporarily frozen for future implantation.

Post-operative treatment is similar for all of the above techniques. A period of non-weight bearing of 3-6 weeks is needed to allow the graft to become more secure within the defect. At least partial range of motion is begun very early in the post-operative period. The patient is gradually progressed to walking and running over the ensuing months.

### EXAMPLES

The following examples serve to illustrate the present invention and are not intended to limits its scope in any way.

### Example 1

A 29-year-old male with knee pain post-injury has a cartilage defect in the medial femoral condyle noted on exam with magnetic resonance imaging (MRI). Patient has stem cells harvested from his iliac crest. Cells are isolated and optionally in vitro-expanded by standard culture expansion techniques. Stem cells are placed in a 1.5% ethanol solution. To the 1.5% ethanol solution are added allograft cartilage fragments, 50-250 μm in size, to generate a 5-10% cartilage fragment solution. A 1.5% ethanol concentration is maintained at this time. To this solution are added growth factors, antioxidants and a three-dimensional collagen I scaffold (an alternate matrix material may be used). The mixture is cultured for 2 weeks under standard culturing techniques, whereby a partially synthesized cartilage ECM is produced. Because the medial femoral condyle has a 3 mm thick articular cartilage layer the cultured graft was made to be 4-5 mm thick. The added thickness is recommended in order to compensate for some shrinkage that occurs at the time of graft implantation. After 2 weeks the partially synthesized graft is inserted into the cartilage defect immediately after fibrin is placed into the defect through a mini-arthrotomy approach. Optionally, a patch is used, with or without fibrin. Optionally, some of the original cells, which have been frozen and saved, are mixed at the time of surgery with a 5-10% cartilage fragment solution and a growth factor. After the cartilage implant is inserted, this mixture is added to fibrin. The cell-cartilage fragment-growth factor-fibrin construct is immediately placed at the implant-defect border. Post-operative management includes range of motion exercises begun within the first 1-2 weeks. Weight bearing is begun at 6-8 weeks. No running is allowed for 6-12 months. The presence of a joint effusion and MRI follow-up exams guide the rate of activity progression.

### Example 2

An 18-year-old female sustains a patellar dislocation and a large chondral fracture off of her medial patellar facet with loose body formation. She has pain and requires surgery. She prefers that only one surgical procedure is performed. She further prefers that allograft tissue is not used. At the time of surgery stem cells are obtained from the iliac crest and isolated utilizing procedures that are known in the art. (U.S. Pat. No. 6,200,606 describes a manner by which to isolate precursor cells, which then may be used in a single stage cartilage repair procedure without the need for in vitro culturing.) The loose body fragment of cartilage is retrieved at the time of surgery. It is grated and cut into small fragments. (Optionally, when available, the fragment may be frozen and pulverized to generate the cartilage fragments, 50 μm-1 mm in size.) While the surgical procedure is being performed, the isolated stem cells are bathed in a 1.5% ethanol solution. Once the cartilage fragments are formed and the cartilage defect is prepared to accept a graft, the cells are then mixed with the cartilage fragments. This mixture is added to an artificial scaffold, i.e., hyaluronan-based, a collagen I or synthetic polylactide scaffold material. Within this scaffold are added a growth factor, a superoxide dismutase-active analogue, ascorbic acid, and minerals. The mixture is stabilized with fibrin and placed into the cartilage defect. Post-operative care includes a brief period of immobilization. Stair climbing is avoided for 6-8 weeks. Activity is progressed based on joint effusion and follow-up MRI exams.

### Example 3

A 35-year-old male with knee pain is found to have a large osteochondral defect on MRI exam. He prefers that the defects are treated with a single surgical procedure, but prefers that the iliac crest harvesting is not done and that allograft cells are not used. It is chosen to treat his defects with an acellular implant. At the time of surgery the defects are prepared to accept a graft. A composite of polylactide-co-glycolide, calcium sulfate, and polyglycolide fibers (the PolyGraft; OsteoBiologics, San Antonio, Tex.) is chosen as the implant graft material. The material is porous. Prior to implantation of the graft, allograft cartilage fragments, 50-250 μm in size, are inserted or pressed into the porous graft into its superficial (cartilage side) surface up to 3 mm in depth. This construct is then inserted into the bone and cartilage defect. A composite of cartilage fragments and fibrin is placed over the defect and across the implant-cartilage defect border.

### Example 4

A 42-year-old female is found to have a large defect on her femoral trochlea and patellar medial facet, as well as her medial femoral condyle. The total surface area of defects is 30 cm². Due to the large defect area, which requires a large amount of cells to be harvested, allograft tissue is chosen for use. Allograft MSCs and allograft cartilage fragments, 50-250 μm in size, are chosen. The MSCs are first contacted with a 1.5% ethanol solution. They are then mixed with the cartilage fragments. This is then added to a three-dimensional scaffold, such as collagen I. An in vitro-culturing procedure is then begun for 1-2 weeks in the presence of a growth factor and under conditions that are favorable for cartilage synthesis. Three grafts are prepared—one that is 8-10 mm thick for the patella, one that is 5-6 mm thick for the trochlear defect, and one that is 4-6 mm thick for the medial femoral condyle. At the time of surgery each implant is inserted into its intended defect. Fibrin is inserted prior to insertion of the implants in order to obtain immediate adhesion of the implants. At the time of surgery some allograft MSCs are mixed with allograft cartilage fragments and a growth factor. Fibrin is then added, and, immediately afterwards, this construct is placed at the borders of the implants and the defect edges. The patient is treated as above. Range of motion is begun within 1-2 weeks. Weight bearing is gradually progressed from 2-6 weeks. Stair climbing is avoided for 6-8 weeks. Activity is progressed based on joint effusion and follow-up MRI exams.

### Example 5

This example describes the preparation of acellular grafts for later implantation.

In this example a collagen I matrix is used as a representative matrix material. A collagen I matrix is mixed with cartilage fragments, which are prepared as described herein. The grafts are pressed into discs that are from 2 to 8 mm thick. Their width can vary from 1×1 cm to 5×5 cm, or more, in size. They are kept frozen for later implantation. When inserted into a cartilage defect, they can be cut to the desired size and shape at the time of surgery. Alternatively, these grafts can be pressed with stem cells at the time of surgery in order to generate a cellular graft. The cells that are pressed into the graft are optionally pre-treated with a dilute alcohol solution.

### Example 6

This example describes in vitro-testing of chondrocytes cultured with cartilage fragments.

Cartilage was collected sterilely from three horses (3-years-old) and freeze-thawed 3 times to ensure all native cells within the cartilage were dead. Prior to the start of the experiment the cartilage was placed in liquid nitrogen and pulverized until it became a fine powder. Then the cartilage was weighed into aliquots to make 2.5% and 10% of cartilage weight to volume in media.

Articular chondrocytes, obtained from cartilage from three horses (3-years-old), were dedifferentiated through monolayer expansion over three weeks. The time at which the monolayers were lifted and returned to non-adherent culture conditions (floaties) is referred to as T0. The lifted cells were maintained in defined, serum-free medium supplemented with ascorbic acid for up to 10 days. The treated cells were co-cultured with 5% (weight/volume) pulverized cartilage (PC) matrix added to the medium.

Collagen type II (Coll II) and aggrecan expression was initially assessed by Northern blot analyses. No mRNA was detectable for either gene in any sample. Follow-up analyses of these genes, and of Coll I mRNA expression, were carried out by real-time quantitative PCR, using Sybr Green fluorescence as the read-out.

Coll II expression increased approximately ten-fold over the first six days after onset of floatie culture conditions, then fell by day 10. The addition of the PC matrix had no obvious effect on Coll II expression, since the patterns and levels of expression were both pretty similar to the control group.

Aggrecan expression was similar to Coll II expression, though perhaps not as marked (5-fold increases, as opposed to 10-fold increases seen in the Col II data).

Collagen type I (Coll I) expression is a marker of chondrocyte dedifferentiation, since differentiated chondrocytes express little if any Coll I transcript. Consistent with this, the T0 level of Coll I expression was around 15 times that measured in the control sample (i.e., articular cartilage). Coll I expression dropped rapidly once the cells were returned to the three-dimensional conditions of the floatie cultures. The addition of the PC to the medium demonstrated a beneficial effect on the rate and extent of Coll I suppression, since Coll I expression fell more rapidly and reached control levels by Day 10, in comparison to the control data.

The experiments were repeated, except that the floatie cultures were maintained for up to 21 days, and the effects of 2.5% and 10% PC matrix were assessed. Coll II expression increased approximately 10-fold over the course of the experiment. The PC had little effect at either concentration. Aggrecan expression also improved over time, and by 21 days, was at levels comparable to that of articular cartilage. PC appeared to have a dose-dependent effect. Coll I expression profiles were also similar to those in AM 1.

When the cartilage fragments were mixed with dedifferentiated chondrocytes, there was rapid binding of the chondrocytes to the borders of the cartilage fragments. These cell-adhered fragments also tended to bind to each other to form rather large, visible clumps. Within one week or so the fragments were no longer visible as they were completely degraded. This indicates that the cartilage fragments induce a rather robust cartilage degradation enzyme expression, such as the metalloproteinases.

### Example 7

This example describes the culture of stem cells with cartilage fragments.

Bone marrow aspirates were obtained from the tuber coxae of 3 normal horses (3-years-old) to attain bone marrow-derived stem cells (MSCs). Aspirates were cultured in media, pre-plated for purification, and grown in monolayer culture flasks for 2-3 weeks until a confluent monolayer culture of MSCs was obtained. Confluent monolayers were expanded for another 2-3 weeks until a minimum of 26×10⁶ cells were attained.

Cartilage was collected sterilely from the same horses and freeze-thawed 3 times to ensure all native cells within the cartilage were dead. Prior to the start of the experiment the cartilage was placed in liquid nitrogen and pulverized until it became a fine powder. Then the cartilage was weighed into aliquots to make 2.5% and 10% of cartilage weight to volume in media.

A 24-well, non-adherent plate contained treatment groups of 1×10⁶ cells only, 1×10⁶ cells with 2.5% cartilage, 1×10⁶ cells with 10% cartilage, 2.5% cartilage only, and 10% cartilage only to make 5 treatment groups. The cells-only treatment group was the baseline positive control. The 2.5% and 10% cartilage fragments without cells served as the negative controls and, if necessary, for baseline values of proteoglycan and DNA content of the matrix provided.

The 5 treatment groups were harvested on day 7 and on day 14 for proteoglycan synthesis, total proteoglycan content, DNA content, and mRNA for aggrecan and collagen type II. For the first horse the MSCs were either immediately combined with the pulverized cartilage or pelleted for 3 days prior to combining with the pulverized cartilage. Both the unpelleted and pelleted MSCs were supplemented with media containing no TGF-β1 or 5 ng/ml of TGF-β1 every other day. MSCs were not pelleted on the following 2 horses based on the first horse's negative results with pelleted MSCs.

The pelleted samples of horse 1 showed no effect with treatment of pulverized cartilage. In all horses proteoglycan synthesis was significantly increased in treatment groups containing cells and cartilage fragments supplemented with 5 ng/ml of TGF-β1. This effect was even more profound by day 14. In fact, in horse 1 the combination of pulverized cartilage and TGF-β1 supplementation appeared necessary for the MSC survival. Again this shows a significant increase in proteoglycan synthesis with MSC treatment of 5 ng/ml of TGF-β1 and pulverized cartilage. For all horses combined this effect was the most profound when MSCs were combined with 2.5% pulverized cartilage.

After combining the data for all horses, the DNA content was significantly increased by treatment with 5 ng/ml of TGF-β1 and pulverized cartilage fragments. By day 14, the predominant effect of significantly increasing DNA content was due to the 5 ng/ml of TGF-β1.

As expected, the total proteoglycan content showed a trend for increasing with an increase in percentage of pulverized cartilage. This is probably due to the large amounts of proteoglycan in native cartilage. This created an extremely high baseline level of proteoglycan that increased with an increase in cartilage concentration. The cells combined with pulverized cartilage were likely lower due to the cells actively remodeling and degrading the matrix. This MCS-mediated degradation of cartilage started at approximately day 2, and matrix resorption became more evident over time. Within one week or so the fragments were no longer visible as they were completely degraded. This indicates that the cartilage fragments induced a rather robust cartilage degradation enzyme expression, such as the metalloproteinases. This is the same effect that is noted in the chondrocytes.

The real-time polymerase chain reaction (RT-PCR) for horse 1 demonstrated, again, that pulverized cartilage and 5 ng/ml of TGF-β1 were necessary for MSC survival. RT-PCR was carried out to ensure the accuracy of the RT-PCR.

Pulverized cartilage enhanced proteoglycan synthesis of MSCs when compared to MSCs alone. Pelleted MSCs were markedly inferior to unpelleted MSCs when combined with pulverized cartilage. TGF-β1 was necessary for MSC survival and chondrogenesis. The optimal amount of pulverized cartilage for MSCs is between 2.5% and 10%.

The pulverized cartilage fragments induced rapid matrix degradation by the induction of matrix-degrading enzymes, such as metalloproteinases, for both chondrocytes and stem cells. The fragments induced the synthesis of cartilage matrix components collagen II and proteoglycan by stem cells, but have little such effect on chondrocytes. This indicates that cartilage matrix turnover is increased by the fragments. It further indicates that the cartilage fragments, in the presence of a growth factor, induce their differentiation into chondrocytic cells, because this effect was not present with either fragments or growth factor alone.

### Example 8

This example describes the culturing of stem cells with a dilute alcohol solution.

Cells are harvested from the tuber coxae of 3 normal horses (3-years-old) to attain bone marrow-derived stem cells (MSCs). Aspirates are cultured in media, pre-plated for purification, and grown in monolayer culture flasks for 2-3 weeks until a confluent monolayer culture of MSCs is obtained. Confluent monolayers are expanded for another 2-3 weeks until a minimum of 26×10⁶ cells are attained.

Alcohol and control mediums are set up. The alcohol treatment media are supplemented with methanol, ethanol, 2-propanol or tert-butanol at concentrations of 0.1 to 3%. Controls have an equal amount of distilled water added. Cultures are then incubated for 72 hours at 37° C. in room air.

Histochemical staining is undertaken for glycosaminoglycan (GAG) and collagen synthesis. Collagen type II (Coll II) and aggrecan expression are assessed by Northern blot analyses. Analyses of these genes, and of Coll I mRNA expression, are carried out by RT-PCR.

Histochemical analysis is expected to reveal a significant increase in GAG and collagen production in those cultures exposed to dilute alcohol solutions. Maximal effects are expected to occur at concentrations of 1.5-2.5% alcohol solutions. Collagen type I expression is expected to be significantly depressed, where collagen type II expression is expected to be increased, in those cultures exposed to the dilute alcohol.

These results are expected to reveal that a dilute alcohol solution induces the articular chondrocyte pathway of differentiation (i.e., increase proteoglycans and collagen II) and depresses or inhibits the hypertrophic pathway (i.e., decreased collagen type I expression).

All references, including publications, patent applications, and patents, cited herein are hereby incorporated by reference to the same extent as if each reference were individually and specifically indicated to be incorporated by reference and were set forth in its entirety herein.

The use of the terms “a,” “an,” “the,” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

What is claimed is:

1. A method of promoting healing of a cartilage defect in a region of cartilage, which comprises the defect and stem cells, which method comprises contacting the region with (i) cartilage fragments, (ii) cartilage fragments and a partially synthesized ECM, or (iii) cartilage fragments, a partially synthesized ECM, and a scaffold, and, optionally, at least one growth factor, whereupon the cartilage fragments induce the stem cells to differentiate into chondrocytes, thereby promoting healing of the cartilage defect.

2. The method of claim 1, which further comprises simultaneously or sequentially, in either order, contacting the region with stem cells, chondrocytes, a proteoglycan, an anti-oxidant, a collagen precursor, a vitamin, a mineral, and/or a cartilage-degrading enzyme.

3. The method of claim 1, wherein the cartilage fragments are stabilized by a biological glue.

4. A method of promoting healing of a cartilage defect in a region of cartilage, which comprises the defect and an implant comprising cartilage scaffold, which method comprises contacting the region with (i) cartilage fragments, (ii) cartilage fragments and a partially synthesized ECM, or (iii) cartilage fragments, a partially synthesized ECM, and a scaffold, and, optionally, at least one growth factor, whereupon the cartilage fragments promote degradation of the cartilage scaffold in the implant, thereby promoting healing of the cartilage defect.

5. The method of claim 4, which further comprises contacting the region with stem cells, chondrocytes, a proteoglycan, an anti-oxidant, a collagen precursor, a vitamin, a mineral, and/or a cartilage-degrading enzyme.

6. The method of claim 4, wherein the cartilage fragments are stabilized by a biological glue.

7. A method of promoting healing of a cartilage defect in a region of cartilage, which comprises the defect, which method comprises (i) contacting the region with an implant comprising cartilage scaffold and cartilage fragments, or (ii) simultaneously or sequentially, in either order, contacting the region with (a) (i′) cartilage fragments, (ii′) cartilage fragments and a partially synthesized ECM, or (iii′) cartilage fragments, a partially synthesized ECM, and a scaffold, and, optionally, at least one growth factor, and (b) an implant comprising cartilage scaffold, whereupon the cartilage fragments promote degradation of the cartilage scaffold in the implant, thereby promoting healing of the cartilage defect.

8. The method of claim 7, which further comprises contacting the region with stem cells, chondrocytes, a proteoglycan, an anti-oxidant, a collagen precursor, a vitamin, a mineral, and/or a cartilage-degrading enzyme.

9. The method of claim 7, wherein the cartilage fragments are stabilized by a biological glue.

10. A method of promoting healing of a cartilage defect in a region of cartilage, which comprises the defect and an implant comprising a cartilage graft, which method comprises contacting the region with (i) cartilage fragments, (ii) cartilage fragments and a partially synthesized ECM, or (iii) cartilage fragments, a partially synthesized ECM, and a scaffold, and, optionally, at least one growth factor, whereupon the cartilage fragments promote incorporation of the cartilage graft into adjacent cartilage in the region, thereby promoting healing of the cartilage defect.

11. The method of claim 10, which further comprises contacting the region with stem cells, chondrocytes, a proteoglycan, an anti-oxidant, a collagen precursor, a vitamin, a mineral, and/or a cartilage-degrading enzyme.

12. The method of claim 10, wherein the cartilage fragments are stabilized by a biological glue.

13. A method of promoting healing of a cartilage defect in a region of cartilage, which comprises the defect, which method comprises (i) contacting the region with an implant comprising a cartilage graft and cartilage fragments, or (ii) simultaneously or sequentially, in either order, contacting the region with (a) (i′) cartilage fragments, (ii′) cartilage fragments and a partially synthesized ECM, or (iii′) cartilage fragments, a partially synthesized ECM, and a scaffold, and, optionally, at least one growth factor, and (b) an implant comprising cartilage scaffold, whereupon the cartilage fragments promote incorporation of the cartilage graft into adjacent cartilage in the region, thereby promoting healing of the cartilage defect.

14. The method of claim 13, which further comprises contacting the region with stem cells, chondrocytes, a proteoglycan, an anti-oxidant, a collagen precursor, a vitamin, a mineral, and/or a cartilage-degrading enzyme.

15. The method of claim 13, wherein the cartilage fragments are stabilized by a biological glue.

16. A method of causing stem cells to differentiate by the articular chondrocyte pathway, which method comprises contacting the stem cells with a compound comprising an active alcohol moiety, whereupon the stem cells differentiate by the articular chondrocyte pathway.

17. The method of claim 16, wherein the compound is selected from the group consisting of methanol, ethanol, propanol, tert-butanol, or a pharmacologically active salt thereof, alone or in combination with a carrier therefor.
